(19) United States
(12) Patent Application Publication
(10) Pub. No.: US 2020/0254211 A1
(43) Pub. Date: Aug. 13, 2020

(54) STRESS RELIEVING DEVICE FOR VEHICLES

(71) Applicants: DENSO CORPORATION, Kariya-city, Aichi-pref. (JP); MIE UNIVERSITY, Tsu-shi, Mie (JP)

(72) Inventors: Shinji AOKI, Kariya-city (JP); Teruhisa KOMORI, Tsu-shi (JP)

(73) Assignees: DENSO CORPORATION, Kariya-city, Aichi-pref. (JP); MIE UNIVERSITY, Tsu-shi, Mie (JP)

(21) Appl. No.: 15/774,311

(22) PCT Filed: Oct. 21, 2016
(86) PCT No.: PCT/JP2016/081196

§ 371 Date: May 8, 2018

(30) Foreign Application Priority Data
Nov. 12, 2015 (JP) 2015-222318

(51) Int. Cl. A61M 21/02; A61M 11/04; A61K 31/045; A61K 47/10; A61K 9/00; B60H 3/00

(52) U.S. Cl. CPC A61M 21/02; A61M 11/04; A61K 31/045; A61B 5/165; A61K 9/0073; B60H 3/0035; A61K 47/10

(57) ABSTRACT

A stress relieving device for vehicles includes a supply unit that sprays cedrol into a vehicle cabin, a detection unit that detects an irritation level and a sleepiness level of a passenger in the vehicle cabin, and a controller that operates the supply unit based on the irritation level detected by the detection unit to cause the cedrol to be volatilized into the vehicle cabin and operates the supply unit to reduce a volatilization amount of the cedrol when the detection unit detects sleepiness. When cedrol is volatilized to suppress irritation in the passenger, a relaxation effect is exhibited, but when relaxation is exceed to promote sleepiness, the volatilization amount of cedrol is reduced to suppress sleepiness.

### CROSS REFERENCE TO RELATED APPLICATIONS

The present application is based on Japanese Patent Application No. 2015-222318 filed on Nov. 12, 2015, the content of which is incorporated herein by reference.

### TECHNICAL FIELD

The present disclosure relates to a stress relieving device for vehicles, which is capable of both relieving stress and suppressing sleepiness with respect to drivers during vehicle operation.

### BACKGROUND ART

Conventional mixture solvents, such as that described in Patent Literature 1, are known. The mixture solvent (liquid composition) in Patent Literature 1 includes sesquiterpene alcohol as a hardly volatile component in a solvent of fluorine-containing ether oil which is perfluoroalkylether or perfluoropolyether, and a value of (solubility parameter of fluorine-containing ether oil)−(solubility parameter of sesquiterpene alcohol) is between −8 to 0. It is described that cedrol may be preferably used as a sesquiterpene alcohol.

The above described mixture solvent may applied to fragrances or perfumes used in, for example including skin care cosmetics, skin cosmetics, hair cosmetics, bath agents, etc., for stable dissolution of volatile compounds and to promote volatilization thereof. The aroma component of fragrances and perfumes are described as having relaxation effects.

### PRIOR ART LITERATURES

### Patent Literature

Patent Literature 1: JP 4824290

### SUMMARY OF INVENTION

The present inventors studied the use of cedrol, which exhibits the above described relaxation effects, in a stress relieving device for vehicles. In other words, by vaporizing cedrol into a vehicle cabin, the stress of a passenger, and especially that of a driver, may be mitigated. However, there is a concern that when increasing the vaporization amount of cedrol, by going beyond a relaxed state, sleepiness may be promoted instead.

An object of the present disclosure is, in view of the above issue, provide a stress relieving device for vehicles capable of providing a relaxation effect to a passenger of a vehicle while at the same time suppressing the onset of sleepiness.

For achieving the above objects, the following technical measures are used.

According to a present disclosure, a stress relieving device for vehicles includes a supply unit that sprays cedrol into a vehicle cabin, a detection unit that detects an irritation level and a sleepiness level of a passenger in the vehicle cabin, and a controller that operates the supply unit based on the irritation level detected by the detection unit to cause the cedrol to be volatilized into the vehicle cabin and operates the supply unit to reduce a volatilization amount of the cedrol when the detection unit detects sleepiness. When cedrol is volatilized to suppress irritation in the passenger, a relaxation effect is exhibited, but when relaxation is exceed to promote sleepiness, the volatilization amount of cedrol is reduced to suppress sleepiness.

According to the above, based on an irritation level of a passenger from the detection unit, the controller volatilizes cedrol from the supply unit into the vehicle cabin, thereby suppressing the irritation of the driver and exhibiting a relaxation effect.

Further, as the volatilization amount of cedrol increases, there is a chance that by exceeding the relaxed state, sleepiness may be promoted. As such, the controller decreases the volatilization amount of cedrol to regulate concentration within the vehicle cabin when sleepiness in the passenger is detected from the detection unit, and it is possible to suppress the manifestation of sleepiness in the passenger.

Further, cedrol is odorless and non-fragrant, so the stress relieving device for vehicles may be used without considering the preferences of passengers.

### BRIEF DESCRIPTION OF DRAWINGS

FIG. 1 is an explanatory drawing showing a stress relieving device for vehicles while mounted in a vehicle.

FIG. 2 is an explanatory drawing showing the configuration of a stress relieving device for vehicles according to a first embodiment.

FIG. 3 is a graph showing activity voltages of α-II waves and θ waves in the brain with respect to cedrol concentration.

FIG. 4 is a graph showing parasympathetic nerve state (relaxed, sleepy state) with respect to cedrol concentration.

FIG. 5 is an explanatory drawing showing a stressed state and a stress reliving state in a vehicle cabin.

FIG. 6 is a graph showing the effects of cedrol with respect to stress.

FIG. 7 is a graph showing decrease in consciousness level and ratio of vicinity to center line during driving.

FIG. 8 is an explanatory drawing showing the configuration of a stress relieving device for vehicles according to a second embodiment.

FIG. 9 is an explanatory drawing showing the configuration of a stress relieving device for vehicles according to a third embodiment.

### DESCRIPTION OF EMBODIMENTS

Hereinafter, a plurality of embodiments for implementing the present disclosure will be described with reference to the figures. Further, in each of the following embodiments, portions which are the same or equivalent to previous embodiments will be denoted with the same reference numerals, and explanations thereof may be omitted for brevity. Further, in each embodiment, if only a portion of components are explained, regarding the other portion of components, the components explained in previous embodiments may be used. In the following embodiments, as long as no particular problems exist, the various embodiments may be partially combined with each other even if a combination is not explicitly described.

### First Embodiment

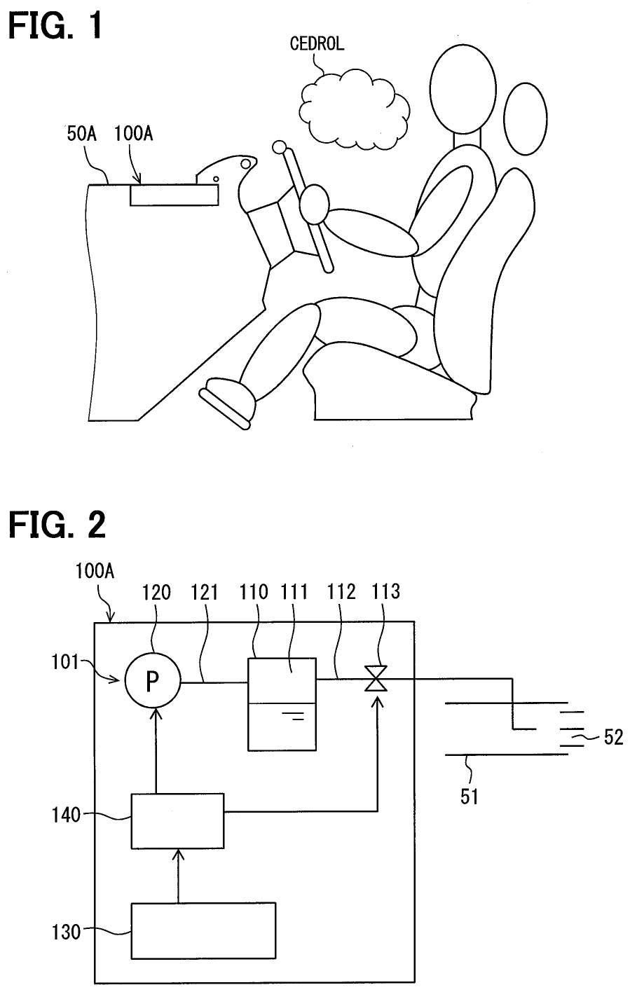

A stress relieving device for vehicles 100A of the first embodiment will be explained with respect to FIGS. 1 to 6. the stress relieving device for vehicles 100A (hereinafter, stress relieving device) is a device that, by cedrol, provides stress relief to a passenger (driver) of a vehicle while at the same time suppressing the onset of sleepiness.

The stress relieving device 100A, as shown in FIGS. 1 and 2, is mounted in an instrument panel 50A of a vehicle that includes an air conditioning device, and includes a supply unit 101 (a bottle 110, a pump 120), a detection unit 130, a controller 140, etc.

The supply unit 101 is configured to spray and vaporize cedrol into the vehicle cabin, and includes a bottle 110, a pump 120, etc.

The bottle 110 is a container that stores cedrol therein. Cedrol is in a mixture solvent form as will be explained later. In the bottle 110, a mixture solvent of cedrol is stored until a mid level, and on the upper side of the liquid surface of the mixture solvent, a space (a chamber 111) filled with a vapor of the solve mixture is provided. The bottle 110 is configured to be periodically refillable with mixture solvent.

A pipe 112 is connected to one end of the chamber 111, and the leading end of this pipe 112 is disposed so as to face toward the vehicle cabin. For example, the leading end of the pipe 112 may be disposed so as to face toward the vehicle cabin through a side face blowout opening 52 in a side face blowout duct 51 among the blowout ducts of a vehicle air conditioning device. Further, the side face blowout opening 52 is provided on the left or ride side of the vehicle, at a position in the instrument panel 50A toward the driver, and is a blowout opening that opens toward the upper half body, and primarily the face, of the driver.

Further, an valve 113 configured to open and close the pipe 112 is disposed at an intermediate part of the pipe 112. The valve 113 is configured to close when the stress relieving device 100A is stopped, and is configured to open when the stress relieving device 100A is in operation. The opening and closing of the valve 113 is controlled by the controller 140 which will be described later.

Here, the mixture solvent of cedrol stored in the bottle 110 is a solvent formed by mixing diethylene glycol monobutyl ether, which is a glycol based organic compound, in cedrol, which is a type of sesquiterpene alcohol.

Cedrol may, for example, be extracted at high purity from cedar oil of coniferous tress such as cedar and cypress. Cedrol exhibits effects of lowering heat rate, respiratory rate, blood pressure, etc. in humans, and also increasing α-II waves in the brain. Further, predominantly in the parasympathetic nervous system, cedrol functions to reduce irritability etc. in humans, thereby providing a relaxation effect.

Diethylene glycol monobutyl ether has good compatibility with cedrol, and at room temperature, functions as a solvent for efficiently volatilizing cedrol.

The mixture solvent of cedrol is a solution with high mutual solubility and volatility in a room at normal air conditioned temperatures. Here, room temperature refers to standard temperatures of a room, for example, from about 15° C. to 25° C. (or approximately 20° C.).

The mixture ratio of cedrol with respect to diethylene glycol monobutyl ether is set to, for example, between 10% to 50% in order to get a significant volatilization ratio of cedrol. To favorably obtain a volatilization ratio of cedrol, the mixture ratio of cedrol in the mixture solve is preferably from 10% to 40%, and even more preferably from 20% to 40%.

The pump 120 is configured to release the vapor inside the chamber 111 from the side face blowout opening 52 into the vehicle cabin via the pipe 112. The discharge side of the pump 120 is connected to the other end of the chamber 111 through a pipe 121. The operation of the pump 120 is controlled by the controller 140 which will be described later.

The detection unit 130 is configured to detect an irritation level and sleepiness level of the driver. Here, irritation in a driver while driving may manifest when, for example, stuck in traffic, when cut off by another driver, etc. Further, sleepiness in a driver while driving may manifest when, for example, lacking sleep or fatigued, and also when too relaxed.

The detection unit 130 may, for example, be configured to detect biological signals of the driver, such as electrocardiograph (ECG), heart rate, brain waves, etc., as the irritation level and sleepiness level. For example, the detection unit 130 may be disposed in the steering wheel, and implemented as a steering wheel ECG device that measures the ECG or heart rate of the driver, implemented as a brain wave device that detects the brain waves of the driver, etc. The biological signals obtained by the detection unit 130 are output to the controller 140 as will be described later. Further, an ECG device or the like is not limited to being mounted in the steering wheel, and may be disposed on a seat surface or seat back, or the like.

Further, instead of a steering wheel ECG device or a brain wave device as described above, a steering sensor, a brake sensor, an accelerator sensor, or a camera, etc., may be used as the detection unit 130 instead. For example, the irritation level and sleepiness level of a driver may be detected based on hasty or sluggish operation with respect to steering speed by a steering sensor, depression speed of brakes by a brake sensor, depression speed of an accelerator pedal by an accelerator sensor, etc. In addition, the irritation level and sleepiness level of a driver may also be detected by using biological signals from the facial image of a driver taken by a camera (e.g., stressed expressions, blinking, eye movements). Hereinafter, the signals detected by the detection unit 130 will be collectively referred to as a detection signal.

The controller 140 is configured to determine the irritation level and sleepiness level of a driver based on detection signals (i.e., biological signals, or other alternative signals) detected by the detection unit 130. The controller 140 is configured to then, according to this irritation level and sleepiness level, control the opening and closing of the valve 113 as well as the operation of the pump 120 so as to control a volatilization amount when volatilizing vapor of the mixture solvent in the bottle 110 into the vehicle cabin (details below).

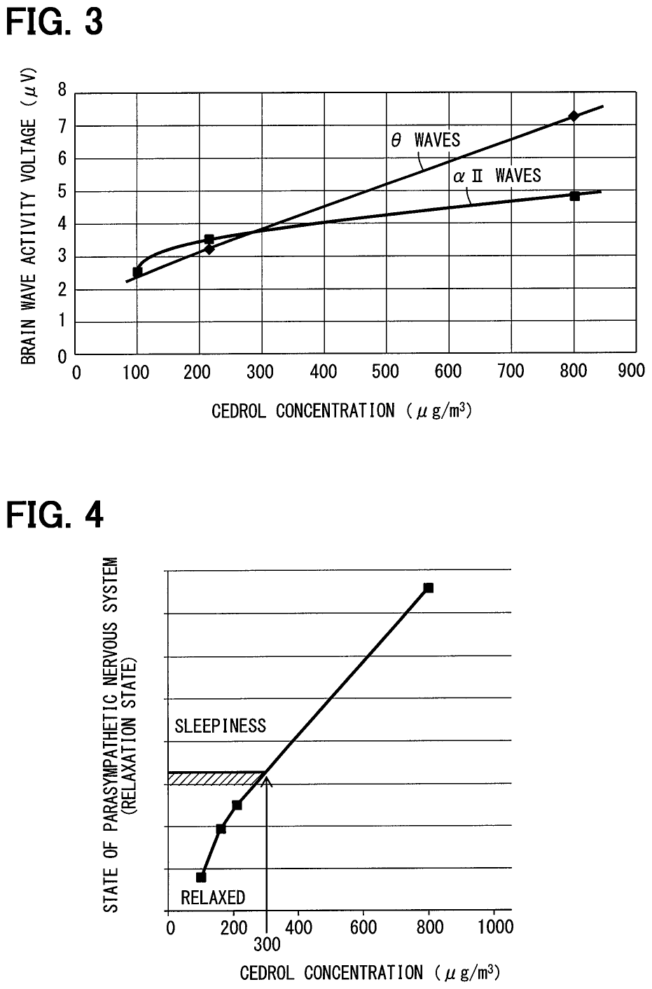

The operation of the stress relieving device 100A with the above described structure will be explained with reference to FIGS. 3 to 6.

First, cedrol is typically known to have relaxation effects with respect to humans (passengers), but the present inventors recognized that cedrol exhibits the functions as shown in FIGS. 3 and 4. In other words, as shown in FIG. 3, in an environment where cedrol is volatilized, when human brain waves are recorded, activity voltages of α-II waves which represent relaxation and θ waves which represent falling asleep both increase as the concentration of cedrol increases.

Further, from the start of volatilization of cedrol and until cedrol concentration reaches 300 μg/m³, the activity voltage of α-II waves is greater than that of θ waves, and it is understood that this is suitable for relaxing a person. Further, once cedrol concentration exceeds 300 μg/m³, the activity voltage of 0 waves is greater than that of α-II waves, and it is understood that this is suitable for promoting sleepiness in a person.

In other words, as shown in FIG. 4, as cedrol concentration increases, parasympathetic nerves become significant, and the state of parasympathetic nerves with respect to cedrol concentration may be considered to be a relaxation state. Then, once cedrol concentration exceeds 300 μg/m³, due to excessive relaxation, sleepiness is promoted.

In the present embodiment, the controls of the stress relieving device 100A are performed under consideration of the above described characteristics of cedrol.

The stress relieving device 100A is preferably used in an environment where the indoor temperature is air conditioned by a vehicle air conditioning device (normal temperatures around 20° C.). The stress relieving device 100A is operated along with an ignition switch of the vehicle being turned on. At the initialization stage, the valve 113 is closed and the pump 120 is stopped, while the detection unit 130 and the controller 140 are turned on.

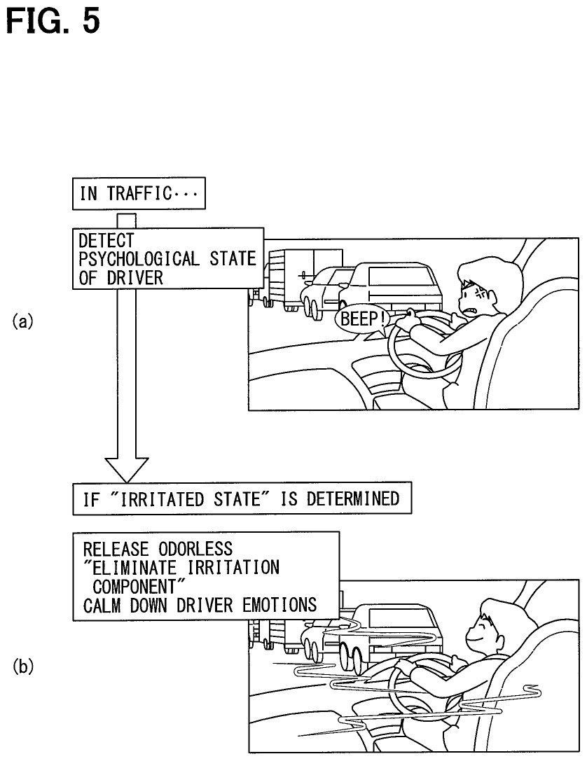

The detection unit 130 periodically detects, with a particular period, a detection signal of biological signals, etc., related to a driver, and then outputs that result to the controller 140. The controller 140 determines that an irritation level of the driver is high when the value of the received detection signal exceeds a predetermined fixed value. For example, as shown in FIG. 5 (a), when a driver feels irritated due to traffic conditions etc., the value of the detection signal increases.

At this time, the controller 140 opens the valve 113 and causes the pump 120 to operate. As a result, due to the discharge pressure of the pump 120, vapor of the mixture solve inside the chamber 111 is released through the pipe 112, the valve 113, and the side face blowout opening 52 into the vehicle cabin, then reaches the upper half body of the driver including the face area. The volatilization amount of the cedrol is preferably from 0.5 to 300 μg/m³. As shown in FIG. 5 (b), the vapor of the mixture solvent (cedrol) emitted toward the driver causes the irritated driver to relax.

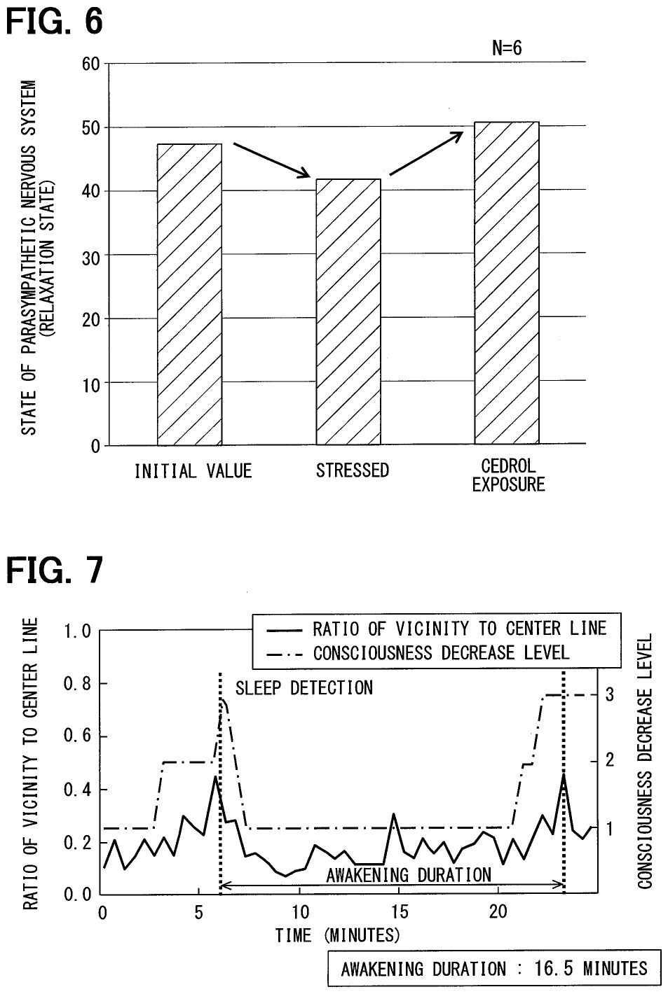

FIG. 6 is the results of an experiment which shows the relaxation effects exhibited by the cedrol of the mixture solvent of the present disclosure. In this experiment, instead of actual driving, a stress substitution experiment is applied to confirm effects. The stress substitution experiment may be, for example, repetitively performing simple calculations, handling heavy objects with difficult posture, etc. FIG. 6 shows the average value of results with respect to six test subjects.

In the above stress substitution experiment, with respect to an initial state, the parasympathetic nerve state (relaxation state) of the test subjects decreases when stressed. Further, when the mixture solvent vapor of the present disclosure (cedrol) is released to the test subjects, it is confirmed that the parasympathetic nerve state increases to above the initial state. In other words, it is possible for a mixture solvent of cedrol is change an irritated state to a relaxed state.

After a passenger is relaxed with cedrol, if cedrol is continuously volatilized, cedrol concentration would increase and in this case sleepiness may be promoted. Accordingly, when the controller 140 determines that a sleepiness level of the driver is high based on the detection signal of biological signals, etc., which is continuously detected from the detection unit 130, the controller 140 decreases the volatilization amount of cedrol. In order to decrease the volatilization amount of cedrol, the output of the pump 120 may be decreased, or the pump 120 may be stopped.

Thereafter, the controller 140 repeatedly performs controls whereby when an irritation level of the driver is detected, then cedrol is volatilized based on this irritation level, and when sleepiness is detected, the volatilization amount of cedrol is decreased in order to regulate concentration in the vehicle cabin.

According to the present embodiment as described above, based on an irritation level of a passenger from the detection unit 130, the controller 140 volatilizes cedrol from the supply unit 101 into the vehicle cabin, thereby suppressing the irritation of the driver and exhibiting a relaxation effect.

Further, as the volatilization amount of cedrol increases, there is a chance that by exceeding the relaxed state, sleepiness may be promoted. As such, the controller 140 decreases the volatilization amount of cedrol when sleepiness in the passenger is detected from the detection unit 130, and it is possible to suppress the manifestation of sleepiness in the passenger.

By relieving stress and suppressing sleepiness during driving, a driver may concentrate on driving and maintain safe driving.

Further, cedrol is odorless and non-fragrant, so the stress relieving device 100A may be used without considering the preferences of passengers.

In addition, a lower limit of cedrol for exhibiting a meaningless relaxation effect by cedrol is set at 0.5 μg/m³. Further, when cedrol volatilization amount exceeds 300 μg/m³, θ waves which signify falling asleep becomes more prominent than α-II waves which signify relaxation (see FIG. 3). Due to this, the upper limit of volatilization amount of cedrol is preferably set to be equal to or under 300 μg/m³.

### Modified Example of First Embodiment

In the above described first embodiment, the sleepiness level of the driver is determined based on detection signals of biological signals etc., but as shown in FIG. 7, the sleepiness level of the driver may be determined based on a distance between the vehicle and the center line during driving instead.

For example, by providing a camera outside of the vehicle that captures the centerline, the controller 140 may determine sleepiness based on a line separation distance between the present vehicle and the center line. The controller 140 determines that the driver is driving awake and not sleepy when the line separation distance is equal to or above a particular value. Further, when the line separation distance is small due to the vehicle approaching or crossing over the traffic lane line, and a ratio of vicinity to the center line is high, the controller 140 determines that the driver is sleepy. In FIG. 7, “consciousness decrease level” is a value that when greater, represents a greater level of sleepiness.

The controller 140, during a state of volatilizing cedrol to suppress an irritation level in the driver, decreases the volatilization amount of cedrol when determining a sleepiness level in the driver from the above described line separation distance. Due to this, the same effects as the above described first embodiment may be exhibited.

Further, as methods of suppressing sleepiness in the driver, in addition to decreasing the volatilization amount of cedrol, there are methods such as temporarily increasing seat belt tension, lower the temperature of the output of an air conditioning device, etc.

### Second Embodiment

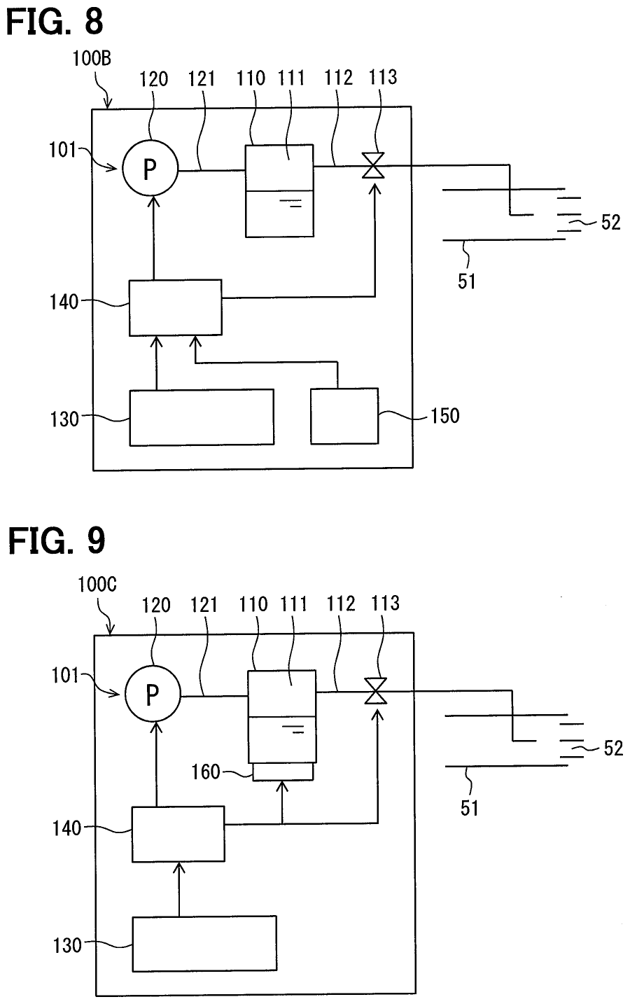

A stress relieving device 100B of a second embodiment is shown in FIG. 8. The second embodiment, when compared to the first embodiment described above, introduces an input unit 150.

The input unit 150 may be, for example, an input switch configured for driver input disposed in the instrument panel 50A near the driver seat. The input unit 150 is configured to receive an input operation from a driver when feeling irritated, and outputs this signal representing irritation to the controller 140.

The controller 140, when receiving the signal from the input unit 150, operates the supply unit 101 to volatilize cedrol into the vehicle cabin regardless of the detection signal of biological signals etc. from the detection unit 130. In other words, according to the present embodiment, cedrol may be volatilized based on request from the driver.

Further, the controller 140 is configured to, based on the signal from the input unit 150, learn and update a determination reference when determining the irritation level of the driver from the detection signal obtained by the detection unit 130. For example, when a signal is input from the input unit 150 even when the detected irritation level is below a standard determination reference, then thereafter, the determination reference is overwritten with the irritation level at that time point.

Due to this, when a passenger feels irritated, cedrol may be intentionally volatilized into the vehicle cabin. Further, the determination reference for irritation level when volatilizing cedrol may be matched with the preferences of a passenger, thereby improving convenience with respect to the passenger.

### Third Embodiment

A stress relieving device 100C of a third embodiment is shown in FIG. 9. The third embodiment, when compared to the first embodiment described above, introduces a heating unit 160.

Operation of the heating unit 160 is controlled by the controller 140. The heating unit 160 is configured to heat the cedrol in the supply unit 101, and may be, for example, an electric heater.

Due to this, by heating cedrol with the heating unit 160, the volatilization ratio of cedrol may be increased to increase the volatilization amount of cedrol, and is effective when the volatilization amount of cedrol into the vehicle cabin is to be increased.

What is claimed is:

1. A stress relieving device for vehicles, comprising:
a supply unit that sprays cedrol into a vehicle cabin,
a detection unit that detects an irritation level and a sleepiness level of a passenger in the vehicle cabin, and
a controller that
operates the supply unit based on the irritation level detected by the detection unit to cause the cedrol to be volatilized into the vehicle cabin, and
operates the supply unit to reduce a volatilization amount of the cedrol when the detection unit detects sleepiness in order to regulate concentration within the vehicle cabin.

2. The stress relieving device for vehicles of claim 1, wherein
when the irritation level is detected, the volatilization amount of the cedrol is set from 0.5 to 300 μg/m3.

3. The stress relieving device for vehicles of claim 1, further comprising:
an input unit configured to, when the passenger feels irritated, allow a signal that represents the irritated feeling to be input to the controller, wherein
the controller, based on the signal from the input unit, operates the supply unit to cause the cedrol to be volatilized into the vehicle cabin.

4. The stress relieving device for vehicles of claim 3, wherein
the controller, based on the signal from the input unit, learns and updates a determination reference of the irritation level for causing the cedrol to be volatilized.

5. The stress relieving device for vehicles of claim 1, further comprising:
a heating unit whose operation is controlled by the controller, the heating unit heating the cedrol in the supply unit.

6. The stress relieving device for vehicles of claim 1, wherein
the cedrol is a solvent mixed with diethylene glycol monobutyl ether.
